(19) United States
(12) Patent Application Publication
(10) Pub. No.: US 2025/0027958 A1
(43) Pub. Date: Jan. 23, 2025

(54) DIAGNOSTIC AND THERAPEUTIC METHODS USING HEME AS A BIOMARKER FOR CELLULAR AND TISSUE DAMAGE

(71) Applicant: Beth Israel Deaconess Medical Center, Inc., Boston, MA (US)

(72) Inventors: Leo E. OTTERBEIN, Beverly, MA (US); Rodrigo ALVES DE SOUZA, Boston, MA (US)

(21) Appl. No.: 18/708,920

(22) PCT Filed: Nov. 14, 2022
(86) PCT No.: PCT/US2022/079819

§ 371 Date: May 9, 2024

Related U.S. Application Data
(60) Provisional application No. 63/279,064, filed on Nov. 12, 2021.

(51) Int. Cl. G01N 33/72; A61K 33/00; A61K 38/17

(52) U.S. Cl. CPC G01N 33/72; A61K 33/00; A61K 38/1709; G01N 2800/52

(57) ABSTRACT

Disclosed herein are methods of assessing cellular or tissue damage in a patient: methods of identifying a patient having sterile inflammation, methods of assessing sterile inflammation in a patient. methods of monitoring a patient undergoing a treatment with a therapeutic agent that causes cellular or tissue toxicity. methods of treating or preventing cellular or tissue damage in a patient in need thereof, methods of treating or preventing sterile inflammation in a patient in need thereof. and related uses and kits.

### STATEMENT REGARDING FEDERALLY SPONSORED RESEARCH

This invention was made with government support under Grant No. W81XWH-16-1-0464 awarded by the United States Army. The government has certain rights in the invention.

### BACKGROUND

Cellular and tissue damage caused, e.g., by sterile inflammation or cytotoxic effects of therapeutic agents, can significantly impair health. For example, many chemotherapeutic agents and types of trauma-mediated inflammation (e.g., motor vehicle accidents, traumatic brain injury, and surgery (e.g., joint replacement, open heart surgery, and cardiopulmonary bypass)) can cause cellular and tissue damage. Detecting cellular and tissue damage and injury can be difficult, especially in instances where physical injury may not be visually apparent, e.g., in the context of sterile inflammation (e.g., ischemia, concussions, and the like).

Therefore, improved diagnostic and therapeutic methods for assessing cellular or tissue damage, including from sterile inflammation, are needed.

### SUMMARY OF THE DISCLOSURE

A first aspect of the disclosure features a method of assessing cellular or tissue damage in a subject, the method including: (a) determining a level of heme in a biological sample (e.g., a biological fluid, e.g., cerebrospinal fluid (CSF), urine, wound fluid (e.g., wound drains), lung fluid (e.g., lung wash fluid collected from a bronchoalveolar lavage), abdomen fluid (e.g., ascites or fluid collected from peritoneal lavage), or sweat) obtained from a subject; and (b) identifying the subject as having cellular or tissue damage based on the level of heme in the biological sample from the patent, wherein a level of heme in the biological sample that is at or above a reference level of heme (e.g., a reference level of about 1 μM to about 10 μM, e.g., about 5 μM to about 10 μM, e.g., about 1 μM, about 2 μM, about 3 μM, about 4 μM, about 5 M, about 6 μM, about 7 μM, about 8 μM, about 9 μM, or about 10 μM) indicates that the subject has cellular or tissue damage.

In some embodiments, the subject has experienced cellular or tissue damage. In some embodiments the cellular or tissue damage occurs as a result of an ischemic event, hypofusion/reperfusion injury, exposure to a therapeutic agent (e.g., a chemotherapeutic agent, as defined herein, or a cytotoxic agent, as defined herein, whether administered for acute or chronic treatment), radiation therapy, radiation injury, pancreatitis, physical trauma, such as a concussion, a drug overdose, surgery (e.g., open chest surgery (e.g., cardiopulmonary bypass surgery), hip replacement surgery, plastic surgery, etc.), hypotension, or shock. In some embodiments, the ischemic event comprises a stroke or a transient ischemic attack (TIA). In some embodiments, the hypofusion/reperfusion injury comprises ischemia reperfusion injury (IRI). In some embodiments, the drug overdose is caused by a therapeutic agent that causes cellular or tissue toxicity. In some embodiments, the drug is acetaminophen. In some embodiments, the level of heme in the biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) from the subject indicates the severity of cellular or tissue damage in the subject. In some embodiments, the subject is undergoing a treatment with a therapeutic agent (e.g., a chemotherapy, a radiation therapy, a virus-mediated therapy, or a CRISPR-mediated gene therapy) that causes cellular or tissue toxicity. In some embodiments, the subject is treated with a radiation therapy that causes cellular or tissue toxicity. In some embodiments, the subject experiences a radiation injury that causes cellular or tissue toxicity. In some embodiments, the subject has experienced a blast injury (e.g., due to an explosion), as in the case of a first responder or military or law enforcement service member, or a rotational injury, such as an injury resulting from spinning, as in the case of a ballerina. In other embodiments, the subject has experienced a sports-related injury, such as an impact sustained during a contact sport, such as football, soccer, basketball, hockey, or rugby, or other sport, such as running, tennis, handball, and the like. In an embodiment, an increase in heme levels (e.g., in a biological fluid, such as blood (e.g., plasma or serum), urine, CSF, etc.) may indicate the presence of the sports-related injury, e.g., reflecting the presence of tissue damage in the subject (e.g., a concussion, contusion, sprain, strain, fracture, or joint dislocation).

A second aspect of the disclosure features a method of identifying a subject having sterile inflammation, the method including: (a) determining a level of heme in a biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) obtained from a subject; and (b) identifying the subject as having sterile inflammation based on the level of heme in the biological sample from the patent, wherein a level of heme in the biological sample that is at or above a reference level of heme (e.g., a reference level of about 1 μM to about 10 μM, e.g., about 5 μM to about 10 μM, e.g., about 1 μM, about 2 μM, about 3 μM, about 4 μM, about 5 μM, about 6 μM, about 7 μM, about 8 μM, about 9 μM, or about 10 μM) indicates that the subject has sterile inflammation.

In some embodiments, the subject has experienced cellular or tissue damage. In some embodiments the cellular or tissue damage occurs as a result of an ischemic event, hypofusion/reperfusion injury, exposure to a therapeutic agent (e.g., a chemotherapy or cytotoxic agent), radiation therapy, radiation injury, pancreatitis, physical trauma, such as a concussion, a drug overdose, surgery (e.g., open chest surgery (e.g., cardiopulmonary bypass surgery), hip replacement surgery, plastic surgery, etc.), hypotension, or shock. In some embodiments, the ischemic event comprises a stroke or a transient ischemic attack (TIA). In some embodiments, the hypofusion/reperfusion injury comprises ischemia reperfusion injury (IRI). In some embodiments, the drug overdose is caused by a therapeutic agent that causes cellular or tissue toxicity. In some embodiments, the drug is acetaminophen. In some embodiments, the level of heme in the biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) from the subject indicates the severity of cellular or tissue damage in the subject. In some embodiments, the subject is undergoing a treatment with a therapeutic agent (e.g., a chemotherapy, a radiation therapy, a virus-mediated therapy, or a CRISPR-mediated gene therapy) that causes cellular or tissue toxicity. In some embodiments, the subject is treated with a radiation therapy that causes cellular or tissue toxicity. In some embodiments, the subject experiences a radiation injury that causes cellular or tissue toxicity. In some embodiments, the subject has experienced a blast injury (e.g., due to an explosion), as in the case of a first responder or military or law enforcement service member, or a rotational injury, such as an injury resulting from spinning, as in the case of a ballerina. In other embodiments, the subject has experienced a sports-related injury, such as an impact sustained during a contact sport, such as football, soccer, basketball, hockey, or rugby, or other sport, such as running, tennis, handball, and the like. In an embodiment, an increase in heme levels (e.g., in a biological fluid, such as blood (e.g., plasma or serum), urine, CSF, etc.) may indicate the presence of the sports-related injury, e.g., reflecting the presence of tissue damage in the subject (e.g., a concussion, contusion, sprain, strain, fracture, or joint dislocation).

A third aspect of the disclosure features a method of assessing sterile inflammation in a subject, the method comprising: (a) obtaining a biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) from the subject after administration of a therapeutic agent or a physical injury to the subject in the absence of bleeding; and (b) detecting the level of heme in the biological sample. In some embodiments, the sample is obtained from the subject no more than five hours (e.g., 1, 2, 3, 4, or 5 hours) after administration of a therapeutic agent or a physical injury to the subject in the absence of bleeding. In some embodiments, the sample is obtained from the subject more than five hours (e.g., 5, 6, 7, 8, 9, 10, 11, 12, 13, 14, 15, 16, 17, 18, 19, 20, 21, 22, 23, or 24 hours) after administration of a therapeutic agent or a physical injury to the subject in the absence of bleeding. In some embodiments, the sample is obtained from the subject one or more days (e.g., 1, 2, 3, 4, 5, 6, 7, 8, 9, 10, 11, 12, 13, or 14 days) after administration of a therapeutic agent or a physical injury to the subject in the absence of bleeding.

In some embodiments, the subject has experienced cellular or tissue damage. In some embodiments the cellular or tissue damage occurs as a result of an ischemic event, hypofusion/reperfusion injury, exposure to a therapeutic agent (e.g., a chemotherapy or cytotoxic agent), radiation therapy, radiation injury, pancreatitis, physical trauma, such as a traumatic brain injury (e.g., a concussion), a drug overdose, surgery (e.g., open chest surgery (e.g., cardiopulmonary bypass surgery), hip replacement surgery, plastic surgery, etc.), hypotension, or shock. In some embodiments, the ischemic event comprises a stroke or a transient ischemic attack (TIA). In some embodiments, the hypofusion/reperfusion injury comprises ischemia reperfusion injury (IRI). In some embodiments, the drug overdose is caused by a therapeutic agent that causes cellular or tissue toxicity. In some embodiments, the drug is acetaminophen. In some embodiments, the therapeutic agent is administered acutely or chronically. In some embodiments, the level of heme in the biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) from the subject indicates the severity of cellular or tissue damage in the subject. In some embodiments, the subject is undergoing a treatment with a therapeutic agent (e.g., a chemotherapy, a radiation therapy, a virus-mediated therapy, or a CRISPR-mediated gene therapy) that causes cellular or tissue toxicity. In some embodiments, the subject is treated with a radiation therapy that causes cellular or tissue toxicity. In some embodiments, the subject experiences a radiation injury that causes cellular or tissue toxicity. In some embodiments, the subject has experienced a blast injury (e.g., due to an explosion), as in the case of a first responder or military or law enforcement service member, or a rotational injury, such as an injury resulting from spinning, as in the case of a ballerina. In other embodiments, the subject has experienced a sports-related injury, such as an impact sustained during a contact sport, such as football, soccer, basketball, hockey, or rugby, or other sport, such as running, tennis, handball, and the like. In an embodiment, an increase in heme levels (e.g., in a biological fluid, such as blood (e.g., plasma or serum), urine, CSF, etc.) may indicate the presence of the sports-related injury, e.g., reflecting the presence of tissue damage in the subject (e.g., a concussion, contusion, sprain, strain, fracture, or joint dislocation).

A fourth aspect of the disclosure features method of monitoring a subject undergoing a treatment with a therapeutic agent that causes cellular or tissue toxicity, the method comprising: (a) determining a level of heme in a biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) obtained from the subject at a time point during or after administration of the therapeutic agent; and (b) comparing the level of heme in the biological sample from the subject with a reference level of heme (e.g., a reference level of about 1 μM to about 10 μM, e.g., about 5 μM to about 10 μM, e.g., about 1 μM, about 2 μM, about 3 μM, about 4 μM, about 5 μM, about 6 μM, about 7 μM, about 8 μM, about 9 μM, or about 10 μM), thereby monitoring the subject undergoing treatment with the therapeutic agent. In some embodiments, the therapeutic agent is administered to the subject to acutely or chronically. In some embodiments, the subject has an increase in the level of heme compared to the reference level of heme (e.g., a reference level of about 1 μM to about 10 μM, e.g., about 5 μM to about 10 μM, e.g., about 1 μM, about 2 μM, about 3 μM, about 4 μM, about 5 μM, about 6 μM, about 7 μM, about 8 μM, about 9 μM, or about 10 μM), and the treatment is adjusted (e.g., the dosage of the treatment is decreased or the duration over which the treatment is administered is increased) or stopped.

In any of the foregoing aspects, the therapeutic agent comprises a gene therapy, such as a virus-mediated therapy (e.g., adeno-associated-virus (AAV) vectors, adenovirus vectors, or lentivirus vectors). In some embodiments, the therapeutic agent features a CRISPR-mediated gene therapy (e.g., a gene editor, base editor, or prime editor). In some embodiments, the therapeutic agent comprises a chemotherapeutic agent. In some embodiments, the chemotherapeutic agent comprises an anthracycline or a platinum-based chemotherapeutic agent. In some embodiments, the anthracycline is doxorubicin. In some embodiments, the cellular or tissue toxicity is cardiotoxicity resulting from administration of doxorubicin. In some embodiments, the platinum-based chemotherapeutic agent is cisplatin, carboplatin, or oxaliplatin. In some embodiments, the platinum-based chemotherapeutic agent is cisplatin. In some embodiments, the cellular or tissue toxicity is kidney injury resulting from administration of cisplatin.

A fifth aspect of the disclosure features the method of any of the foregoing aspects, further comprising selecting a therapy for the subject to ameliorate the cellular or tissue damage. In some embodiments, the therapy comprises a heme scavenger therapy, a carbon monoxide (CO) therapy (e.g., HBI-002 or CO-306), or a combination thereof.

A sixth aspect of the disclosure features the method of any one of the foregoing aspects, further comprising administering an effective amount of a heme scavenger therapy, a CO therapy (e.g., HBI-002 or CO-306), or a combination thereof to the subject.

A seventh aspect of the disclosure features method of treating or preventing cellular or tissue damage in a subject in need thereof, the method comprising administering an effective amount of a heme scavenger therapy, a CO therapy, or a combination thereof to the subject, wherein the subject has been determined to have a level of heme in the biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) that is at or above a reference level of heme (e.g., a reference level of about 1 μM to about 10 μM, e.g., about 5 μM to about 10 μM, e.g., about 1 μM, about 2 μM, about 3 μM, about 4 μM, about 5 μM, about 6 μM, about 7 μM, about 8 μM, about 9 μM, or about 10 μM).

In some embodiments, the cellular or tissue damage occurs as a result of an ischemic event, hypofusion/reperfusion injury, exposure to a therapeutic agent (e.g., a chemotherapy or cytotoxic agent), radiation therapy, radiation injury, pancreatitis, physical trauma, such as a concussion, a drug overdose, surgery (e.g., open chest surgery (e.g., cardiopulmonary bypass surgery), hip replacement surgery, plastic surgery, etc.), hypotension, or shock. In some embodiments, the ischemic event comprises a stroke or a TIA. In some embodiments, the hypofusion/reperfusion injury comprises ischemia reperfusion injury (IRI). In some embodiments, the cellular or tissue damage comprises cardiotoxicity, liver toxicity, kidney toxicity, or brain toxicity. In some embodiments, the cellular or tissue damage comprises cardiotoxicity. In some embodiments, the subject is treated with a radiation therapy that causes cellular or tissue toxicity. In some embodiments, the subject experiences a radiation injury that causes cellular or tissue toxicity. In some embodiments, the subject has experienced a blast injury (e.g., due to an explosion), as in the case of a first responder or military or law enforcement service member, or a rotational injury, such as an injury resulting from spinning, as in the case of a ballerina. In other embodiments, the subject has experienced a sports-related injury, such as an impact sustained during a contact sport, such as football, soccer, basketball, hockey, or rugby, or other sport, such as running, tennis, handball, and the like. In an embodiment, an increase in heme levels (e.g., in a biological fluid, such as blood (e.g., plasma or serum), urine, CSF, etc.) may indicate the presence of the sports-related injury, e.g., reflecting the presence of tissue damage in the subject (e.g., a concussion, contusion, sprain, strain, fracture, or joint dislocation).

In some embodiments, the subject is undergoing treatment with a chemotherapeutic agent (e.g., for acute or chronic therapy). In some embodiments, the chemotherapeutic agent is an anthracycline or a platinum-based chemotherapeutic agent. In some embodiments, the anthracycline is doxorubicin. In some embodiments, the heme scavenger therapy, the CO therapy, or the combination thereof attenuates acute effects of doxorubicin treatment, chronic effects of doxorubicin treatment, or both, compared to a control therapy. In some embodiments, the acute effects of doxorubicin treatment comprise an increase in serum creatine kinase levels, heme levels, or both. In some embodiments, the chronic effects of doxorubicin treatment comprise long-term cardiac injury, an increase in a level of one or more cardiac dysfunction markers compared to a reference level of the one or more cardiac dysfunction markers, or both. In some embodiments, the cardiac dysfunction markers comprise atrial natriuretic peptide (ANP), brain natriuretic peptide (BNP), cardiac beta-myosin heavy chain (β-MyHC), or a combination thereof. In some embodiments, the platinum-based chemotherapeutic agent is cisplatin, carboplatin, or oxaliplatin. In some embodiments, the platinum-based chemotherapeutic agent is cisplatin.

In some embodiments, the CO therapy results in an elevated expression level of heme oxygenase-1 (HO-1) in the heart compared to a control therapy. In some embodiments, the heme scavenger therapy comprises hemopexin, haptoglobin, albumin, α1-microglobulin, or α1-antitrypsin. In some embodiments, the heme scavenger therapy comprises hemopexin. In some embodiments, the CO therapy is a low dose CO therapy. In some embodiments, the CO therapy comprises HBI-002 or CO-306, which can be used, e.g., to reduce heme levels or to scavenge excess heme.

In some embodiments, the heme scavenger therapy, the CO therapy, or the combination thereof is administered by any suitable administration route, e.g., orally, by inhalation, intravenously, subcutaneously, topically, or intramuscularly. In some embodiments, the heme scavenger therapy, the CO therapy, or the combination thereof is administered orally.

In some embodiments, the biological sample is a biological fluid. In some embodiments, the biological fluid is a blood (e.g., serum or plasma) sample, a CSF sample, urine, wound fluid, lung fluid (e.g., lung wash fluid collected from a bronchoalveolar lavage), abdomen fluid (e.g., fluid collected from peritoneal lavage), or sweat.

In some embodiments, the blood sample is a whole blood sample, a serum sample, a plasma sample, or a combination thereof. In some embodiments, the blood sample is a serum sample.

In some embodiments, the level of heme is the level of free heme.

In some embodiments, the reference level of heme is a baseline level of heme. In some embodiments, the baseline level of heme is a level of heme in a biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) obtained from the subject prior to the onset of cellular or tissue damage. In some embodiments, the reference level of heme is about 5 μM or above. In some embodiments, the reference level of heme is about 5 μM to about 10 μM.

In some embodiments, the subject has a level of heme of about 1000 μM or above. In some embodiments, the subject has a level of heme of about 1000 μM to about 50,000 μM.

In some embodiments, the biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) is obtained from the subject within about 5 hours from the onset of cellular or tissue damage. In some embodiments, the biological sample is obtained from the subject within about 1 minute to about 1 hour from the onset of cellular or tissue damage. In some embodiments, the biological sample is obtained from the subject within about 30 min from the onset of cellular or tissue damage.

An eighth aspect of the disclosure features a method of treating or preventing sterile inflammation in a subject in need thereof, the method comprising administering an effective amount of a heme scavenger therapy, a CO therapy, or a combination thereof to the subject, wherein the subject has been determined to have a level of heme in the biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) that is at or above a reference level of heme (e.g., a reference level of about 1 μM to about 10 μM, e.g., about 5 μM to about 10 μM, e.g., about 1 μM, about 2 μM, about 3 μM, about 4 μM, about 5 μM, about 6 μM, about 7 μM, about 8 μM, about 9 μM, or about 10 μM), such as a reference level of about 10 μM.

In some embodiments, the cellular or tissue damage occurs as a result of an ischemic event, hypofusion/reperfusion injury, chemotherapy, pancreatitis, a physical injury, such as a concussion, a drug overdose, surgery (e.g., open chest surgery (e.g., cardiopulmonary bypass surgery), hip replacement surgery, plastic surgery, etc.), hypotension, or shock. In some embodiments, the ischemic event comprises a stroke or a TIA. In some embodiments, the hypofusion/reperfusion injury comprises IRI. In some embodiments, the cellular or tissue damage comprises cardiotoxicity, liver toxicity, kidney toxicity, or brain toxicity. In some embodiments, the cellular or tissue damage comprises cardiotoxicity. In some embodiments, the subject has experienced a blast injury (e.g., due to an explosion), as in the case of a first responder or military or law enforcement service member, or a rotational injury, such as an injury resulting from spinning, as in the case of a ballerina. In other embodiments, the subject has experienced a sports-related injury, such as an impact sustained during a contact sport, such as football, soccer, basketball, hockey, or rugby, or other sport, such as running, tennis, handball, and the like. In an embodiment, an increase in heme levels (e.g., in a biological fluid, such as blood (e.g., plasma or serum), urine, CSF, etc.) may indicate the presence of the sports-related injury, e.g., reflecting the presence of tissue damage in the subject (e.g., a concussion, contusion, sprain, strain, fracture, or joint dislocation).

In some embodiments, the subject is undergoing treatment with a chemotherapeutic agent or radiation therapy. In some embodiments, the chemotherapeutic agent is an anthracycline or a platinum-based chemotherapeutic agent. In some embodiments, the anthracycline is doxorubicin. In some embodiments, the heme scavenger therapy, the CO therapy, or the combination thereof attenuates acute effects of doxorubicin treatment, chronic effects of doxorubicin treatment, or both, compared to a control therapy. In some embodiments, the acute effects of doxorubicin treatment comprise an increase in serum creatine kinase levels, heme levels, or both. In some embodiments, the chronic effects of doxorubicin treatment comprise long-term cardiac injury, an increase in a level of one or more cardiac dysfunction markers compared to a reference level of the one or more cardiac dysfunction markers, or both. In some embodiments, the cardiac dysfunction markers comprise atrial natriuretic peptide (ANP), brain natriuretic peptide (BNP), cardiac beta-myosin heavy chain (β-MyHC), or a combination thereof. In some embodiments, the platinum-based chemotherapeutic agent is cisplatin, carboplatin, or oxaliplatin. In some embodiments, the platinum-based chemotherapeutic agent is cisplatin.

In some embodiments, the CO therapy results in an elevated expression level of heme oxygenase-1 (HO-1) in the heart compared to a control therapy. In some embodiments, the heme scavenger therapy comprises hemopexin, haptoglobin, albumin, α1-microglobulin, or α1-antitrypsin. In some embodiments, the heme scavenger therapy comprises hemopexin. In some embodiments, the CO therapy is a low dose CO therapy. In some embodiments, the CO therapy comprises HBI-002 or CO-306.

In some embodiments, the biological sample is a biological fluid (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat). In some embodiments, the biological fluid is a blood sample, a CSF sample, urine, or sweat.

In some embodiments, the blood sample is a whole blood sample, a serum sample, a plasma sample, or a combination thereof. In some embodiments, the blood sample is a serum sample.

In some embodiments, the level of heme is the level of free heme.

In some embodiments, the reference level of heme is a baseline level of heme. In some embodiments, the baseline level of heme is a level of heme in a biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) obtained from the subject prior to the onset of cellular or tissue damage. In some embodiments, the reference level of heme is about 5 μM or above. In some embodiments, the reference level of heme is about 5 μM to about 10 μM.

In some embodiments, the subject has a level of heme of about 1000 μM or above. In some embodiments, the subject has a level of heme of about 1000 μM to about 50,000 μM.

In some embodiments, the biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) is obtained from the subject within about 5 hours from the onset of cellular or tissue damage. In some embodiments, the biological sample is obtained from the subject within about 1 minute to about 1 hour from the onset of cellular or tissue damage. In some embodiments, the biological sample is obtained from the subject within about 30 min from the onset of cellular or tissue damage.

A ninth aspect of the disclosure features a kit for assessing cellular or tissue damage in a subject, the kit comprising: (a) reagents for determining a level of heme in a biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) obtained from a subject; and (b) instructions to identify the subject as having cellular or tissue damage based on the level of heme in the biological sample from the patent, in which a level of heme in the biological sample that is at or above a reference level of heme (e.g., a reference level of about 1 μM to about 10 μM, e.g., about 5 μM to about 10 μM, e.g., about 1 μM, about 2 μM, about 3 μM, about 4 μM, about 5 μM, about 6 μM, about 7 μM, about 8 μM, about 9 μM, or about 10 μM) indicates that the subject has cellular or tissue damage. In some embodiments, the reagents are reagents for flow cytometry (FC), fluorescence-activated cell sorting (FACS), Western blot, enzyme-linked immunosorbent assay (ELISA), mass spectrometry (MS; e.g., electrospray ionization (ESI)-MS (ESI-MS) or matrix-assisted laser desorption/ionization (MALDI)-MS), immunofluorescence (IF), immunoprecipitation (IP), radioimmunoassay, dot blotting, high performance liquid chromatography (HPLC), surface plasmon resonance, optical spectroscopy, or immunohistochemistry (IHC).

A tenth aspect of the disclosure features kit for identifying a subject having sterile inflammation, the kit comprising: (a) reagents for determining a level of heme in a biological sample (e.g., a biological fluid, e.g., CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) obtained from a subject; and (b) instructions to identify the subject as having sterile inflammation based on the level of heme in the biological sample from the patent, in which a level of heme in the biological sample that is at or above a reference level of heme (e.g., a reference level of about 1 μM to about 10 μM, e.g., about 5 μM to about 10 μM, e.g., about 1 μM, about 2 μM, about 3 μM, about 4 μM, about 5 μM, about 6 μM, about 7 μM, about 8 μM, about 9 μM, or about 10 μM) indicates that the subject has sterile inflammation.

In each of the aspects above, the subject is a human.

In each of the above aspects, a subject may have a reference level of heme that is less than about 5 μM (e.g., about 4 μM, about 3 μM, about 2 μM, about 1 M, about 0.5 μM, or less) or about 5 μM to about 10 μM (e.g., about 5 μM, about 6 μM, about 7 μM, about 8 μM, about 9 μM, or about 10 μM).

Other features and advantages of the disclosure will be apparent from the following Detailed Description and the Claims.

### BRIEF DESCRIPTION OF DRAWINGS

The accompanying drawings are included to illustrate embodiments of the disclosure and further an understanding of its implementations.

FIG. 1 is a schematic illustrating the experimental regimen for assessing acute and chronic doxorubicin (DOX) cytotoxicity in mice that are administered HBI-002 or a control vehicle. In brief, for the assessment of acute cytotoxicity, male C57BL/6 mice (10-12 weeks old) are administered HBI-002 (10 ml/kg, per os) or a control vehicle (10ml/kg, per os) at 0 hr and 1 hr, followed by administration of doxorubicin at 1.25 hr (20 mg/kg, intraperitoneal injection) and blood and tissue collection at 2.25 hr. In brief, for the assessment of chronic cytotoxicity, male C57BL/6 mice (10-12 weeks old) are administered HBI-002 (10 ml/kg, per os) or a control vehicle (10 ml/kg, per os) daily, followed by administration of doxorubicin (2 mg/kg, intraperitoneal injection) every two days for a total of ten injections; blood and tissue is collected at 8 weeks.

FIG. 2A shows a quantification of carboxyhemoglobin (COHb) levels in mice following administration of HBI-002, or control. Notably, HBI-002 increases COHb levels in mice. Mean±SEM; *p<0.05, **p<0.01, ***p<0.001. n=3-7

FIG. 2B shows a quantification of Heme Oxygenase-1 (HO-1) mRNA in the heart following administration of HBI-002, or control. Notably, ten days of HBI-002 administration in mice upregulates HO-1 mRNA levels in the heart. Mean±SEM; *p<0.05, **p<0.01, ***p<0.001. n=3-7

FIG. 2C shows a quantification of serum heme levels in mice administered HBI-002, or control. Notably, HBI-002 attenuated the increase in serum heme levels after one hour of a single dose of DOX (20 mg/kg, i.p.). Mean±SEM; *p<0.05, **p<0.01, ***p<0.001. n=3-7

FIG. 2D shows a quantification of serum creatine kinase levels in mice administered HBI-002,or control. Notably, HBI-002 attenuated the increase in serum creatine kinase levels after one hour of a single dose of DOX (20 mg/kg, i.p.). Mean±SEM; *p<0.05, **p<0.01, ***p<0.001. n=3-7

FIG. 2E shows a quantification of the ejection fraction of blood in mice administered HBI-002, or control. Notably, HBI-002 prevented DOX-induced cardiac injury, as measured by the ejection fraction in mice. Mean±SEM; *p<0.05, **p<0.01, ***p<0.001. n=3-7

FIG. 2F shows a quantification of the survival rate in mice administered HBI-002. Notably, HBI-002 significantly mitigated mortality in mice. Mean±SEM; *p<0.05, **p<0.01, ***p<0.001. n=3-7

FIG. 3A shows that HBI-002 attenuated long-term (8 weeks) cardiotoxic effects of DOX. HBI-002 chronic dose regimen (FIG. 1) prevented long-term DOX-induced cardiac injury, as measured by the blood ejection fraction, left ventricle (LV) volume, and fractional shortening. Mean±SEM; *p<0.05, **p<0.01, ***p<0.001. n=3-7 mice/group

FIG. 3B shows a quantification of mRNA cardiac dysfunction markers: ANP (atrial natriuretic peptide), BNP (brain natriuretic peptide), β-MyHC (cardiac beta-myosin heavy chain). Mean±SEM; *p<0.05, **p<0.01, ***p<0.001. n=3-7 mice/group.

FIG. 4A shows a schematic illustrating the experimental regimen for assessing the efficacy of DOX chemotherapy treatment in mice that are also administered HBI-002 or a control vehicle. Briefly, male C57BL/6 mice (10-12 weeks old) were first injected with 10⁶ Lewis lung carcinoma cells, followed by administration of HBI-002 (10 ml/kg, per os) or a control vehicle (10 ml/kg, per os) and doxorubicin (4 mg/kg, intraperitoneal injection) at the indicated timepoints.

FIG. 4B shows that HBL-002 does not affect DOX chemotherapy and promotes cardio protection by HO-1 overexpression in the heart, as shown by the quantification of cell tumors and of the cardiac dysfunction marker atrial natriuretic peptide (ANP) and Heme Oxygenase-1 (HO-1). Mean±SEM; *p<0.05, **p<0.01. n=3-6 mice/group.

FIG. 5 shows the level of plasma heme in humans, pigs, and mice after a traumatic brain injury (TBI). Plasma samples were assayed for heme levels comparing baseline uninjured controls to human (day 1), pigs (30 min) and mice (30 min) after TBI. *p<0.05, **p<0.01, ****P<0.001 vs baseline or healthy volunteers.

FIG. 6 shows a transient increase in heme levels detected in cerebrospinal fluid (CSF) from mice after TBI. CSF was collected at the indicated time points from mice after TBI. *p<0.05.

FIG. 7 shows a transient increase in heme levels detected in plasma from mice after cisplatin administration. Plasma was collected at the indicated time points from mice after cisplatin administration (20 mg/kg, i.p.). *p<0.05.

FIG. 8 shows a transient increase in heme levels detected in plasma from pigs after open chest surgery. Plasma was collected over 1 h during sternotomy and isolation of the left descending artery prior to ligation. *p<0.05 vs baseline (n=4/group).

### DEFINITIONS

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

By “therapeutic agent” is meant any agent that, when administered to a subject, has a therapeutic, and/or prophylactic effect and/or elicits a desired biological and/or pharmacological effect. A therapeutic agent may cause cellular tissue or damage and/or toxicity. In one example, a “therapeutic agent” may be a gene therapy, such as a virus-mediated therapy (e.g., adeno-associated-virus (AAV) vectors, adenovirus vectors, or lentivirus vectors), a CRISPR-mediated gene therapy (e.g., a gene editor, base editor, or prime editor), or a combination thereof. In yet another example, the therapeutic agent may be a chemotherapeutic agent.

A “chemotherapeutic agent” is a type of therapeutic agent that is useful for treatment of cancer or other proliferative disorders. Examples of chemotherapeutic agents include cytotoxic agents; 5-FU (5-fluorouracil); alkylating agents such as thiotepa and CYTOXAN® cyclosphosphamide; alkyl sulfonates such as busulfan, improsulfan and piposulfan; aziridines such as benzodopa, carboquone, meturedopa, and uredopa; a camptothecin (including topotecan and irinotecan); 5α-reductases including finasteride and dutasteride); nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such methotrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; anti-adrenals such as aminoglutethimide, mitotane, and trilostane; an epothilone; etoglucid; gallium nitrate; hydroxyurea; lentinan; lonidainine; maytansinoids such as maytansine and ansamitocins; cyclophosphamide; thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., anthracyclines such as doxorubicin, epirubicin, daunorubicin, etoposide, and bleomycin), and DNA alkylating agents (e.g., tamoxigen, prednisone, dacarbazine, mechlorethamine, cisplatin, methotrexate, 5-fluorouracil, and ara-C); and pharmaceutically acceptable salts, acids, prodrugs, and derivatives of any of the above.

The term “radiation therapy” refers to the treatment of a disease using radiation particles or waves (e.g., x-rays, gamma rays, electron beams, or protons. Radiation therapy may be any form of external or internal radiation therapy. For example, radiation therapy may be external beam radiation therapy (e.g., x-ray or photon radiation), brachytherapy, intraoperative radiation therapy (IORT), or stereotactic radiosurgery (SRS).

The term “cytotoxic agent” as used herein refers to any agent that is toxic to cells (e.g., causes cell death, inhibits proliferation, or otherwise hinders a cellular function). Cytotoxic agents include, but are not limited to, chemotherapeutic agents; radioactive isotopes; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents include anti-microtubule agents, alkylating agents, antibiotic agents, topoisomerase inhibitors (e.g., topoisomerase I and topoisomerase II inhibitors), antimetabolites, hormones and analogs thereof, immunotherapeutic agents, proapoptotic agents, inhibitors of LDH-A, inhibitors of fatty acid biosynthesis, cell cycle signaling inhibitors, proteasome inhibitors, and inhibitors of cancer metabolism. In one instance, the cytotoxic agent is a platinum-based chemotherapeutic agent (e.g., cisplatin, carboplatin, or oxaliplatin).

By “hypotension” is meant a decreased in blood pressure (e.g., a systolic blood pressure of less than 90 mm Hg and/or a diastolic blood pressure of less than 60 mm Hg, or a systolic blood pressure of less than 80 mm Hg and/or diastolic blood pressure of less than 50 mm Hg). Hypotension may also be associated with one of more of the following symptoms: chest pain, shortness of breath, irregular heartbeat, loss of consciousness, and/or seizures.

By “hypofusion/reperfusion injury” is meant damage to tissue that results when an oxygenated blood supply returns to a tissue after a period of ischemia (e.g., period of ischemia greater than 1 minute, 5 minutes, 10 minutes, 30 minutes, 1 hour, 2 hours, 3 hours, 4 hours, 5 hours, or 6 hours). The decrease in oxygen and nutrients provided by blood to a tissue creates a state, in which the restoration of blood to the tissue causes damage through induction of oxidative stress. Hypofusion/reperfusion injury may be caused, for example, by surgery (e.g., open chest surgery (e.g., cardiopulmonary bypass surgery), hip replacement surgery, plastic surgery, etc.) or cardiomyopathy. Hypofusion/reperfusion injury is also commonly referred to as ischemic/reperfusion injury.

By “pancreatitis” is meant the inflammation of the pancreas. The term pancreatitis may refer to acute or chronic pancreatitis. Non-limiting examples of symptoms of pancreatitis include severe abdominal pain, nausea, vomiting, increased heart rate, and increased respiratory rate.

By “sample” or “biological sample” is meant any specimen (e.g., blood, serum, plasma, urine, saliva, wound fluid (e.g., drains), amniotic fluid, cerebrospinal fluid, lung fluid (e.g., lung wash fluid collected from a bronchoalveolar lavage), abdomen fluid (e.g., ascites or fluid collected from peritoneal lavage), tissue (e.g., placental or dermal), pancreatic fluid, chorionic villus sample, and cells) taken from a subject. Preferably, the sample is taken from a portion of the body affected by sterile inflammation (e.g., a sterile systemic inflammation).

By “shock” is meant an inadequate perfusion of blood to a tissue in a subject. Non-limiting symptoms of shock include tachycardia, hypotension, hypoxemia, and tachypnoea.

By “subject” or “patient,” which are used interchangeably herein, is meant any animal (e.g., human, cat, dog, horse, monkey, mouse, rat, and rabbit). The subject is preferably a human.

By “infective” inflammation is meant an inflammation that is caused by an infection of a microbial pathogen such as a bacterium, virus, or fungus. An infective inflammation may be indicated by a microbial (e.g., bacterial) nucleic acid (e.g., 16S DNA or 16S rRNA) concentration of >0.5 μg/ml or >1 μg/mL.

By “sterile inflammation” is meant an inflammation that is not caused by an infection of a pathogen such as a bacterium, virus, or fungus. Causes of sterile infection include, for example, mitochondrial nucleic acid released from cells as a result of trauma (e.g., motor vehicle accidents, traumatic brain injury, and the like) or scheduled surgical trauma (e.g., joint replacement, open heart surgery, cardiopulmonary bypass, and the like). A subject may have an inflammation that has both “infective” and “sterile” etiologies. A sterile inflammation may be indicated by a mitochondrial nucleic acid (cytochrome B mitochondrial DNA) of >1 μg/mL or >0.5 μg/mL. A sterile inflammation may also be indicated by a mitochondrial nucleic acid to microbial (e.g., bacterial) nucleic acid ratio of >1:1000 or >1:800.

By “surgery” is meant an invasive therapeutic procedure. Non-limiting examples of surgery include elective surgery, emergency surgery, exploratory surgery, amputation, replantation, reconstructive surgery, cosmetic surgery, transplantation, angioplastic surgery, laparoscopic surgery, laparotomy, laser surgery, and microsurgery.

By “tissue damage” is meant cellular damage to a tissue in the body of a subject. Tissue damage may occur as a result of blunt trauma, may be induced by one or more (e.g., two, three, or four) disease states in a subject (e.g., hypotension, hypofusion/reperfusion injury, pancreatitis, and shock), or may be induced by therapeutic treatment (e.g., surgery, chemotherapy, or radiation), including by overdoses of therapeutic agents. Tissue damage may also be caused by a chronic disease state in a subject.

Other features and advantages of the disclosure will be apparent from the following description of the various embodiments thereof, and from the claims.

### DETAILED DESCRIPTION

The present disclosure is based, at least in part, on the discovery that heme measured or detected in biofluids (e.g., blood, CSF, urine, and the like) when elevated, directly reflects cellular damage and can be used as a biomarker/diagnostic that can reflect the inherent risk of further inflammation and tissue injury. The data provided herein demonstrate identification of significant increases in heme after exposure to a therapeutic agent and trauma, such as following administration of a chemotherapeutic agent, such as cisplatin and doxorubicin, and after traumatic brain injury (TBI) and surgery. Based on these data, it is expected that any drug designed to destroy cells, whether cancer or normal cells, would be expected to lead to the release of heme. Moreover, drugs, such as acetaminophen, may cause tissue damage (e.g., when administered in excess). Thus, the presence of heme in a biological sample (e.g., a biological fluid, e.g., cerebrospinal fluid (CSF), urine, wound fluid (e.g., wound drains), lung fluid (e.g., lung wash fluid collected from a bronchoalveolar lavage), abdomen fluid (e.g., ascites or fluid collected from peritoneal lavage), or sweat) may reflect the presence of a drug overdose in a subject, the presence or risk of cellular or tissue damage, and/or the occurrence of a prior traumatic event or injury that resulted in cellular or tissue damage.

### Reference Levels

The level of one or more biomarkers (e.g., heme) in a biological sample (e.g., a biological fluid, e.g., cerebrospinal fluid (CSF), urine, wound fluid (e.g., wound drains), lung fluid (e.g., lung wash fluid collected from a bronchoalveolar lavage), abdomen fluid (e.g., ascites or fluid collected from peritoneal lavage), or sweat) obtained from a subject can be compared to a reference level of the biomarker to diagnose the subject, to assess cellular or tissue damage in a subject, to identify a subject having sterile inflammation, to assess sterile inflammation, or to monitor a subject undergoing a treatment with a therapeutic agent that causes cellular or tissue toxicity. Any suitable reference levels may be used. The reference level may be generated using a positive or negative control sample, e.g., the level of one or more biomarker in a subject can be compared directly to the level of one or more biomarker in a negative control sample (e.g., a biological sample from a healthy subject not having cellular or tissue damage or a biological sample obtained from the subject at an earlier timepoint when the subject was diagnosed as not having cellular or tissue damage) or a positive control sample (e.g., a biological sample from a subject clinically diagnosed (e.g., diagnosed using liver biopsy or imaging) with cellular or tissue damage or sterile inflammation). Data (e.g., the level of one or more biomarker in a sample from a subject) collected from the subject (e.g., raw data) can be compared to a reference level (e.g., the level of one or more biomarkers in a sample from a positive or negative control). The reference level may be based on data from a single subject (e.g., a single positive or negative control) or from a population of such subjects.

The subject may also be diagnosed by comparing data from the subject (e.g., a level of one or more biomarker (e.g., heme)) to transformed data, such as data that have been transformed using a machine learning algorithm(s) to produce a reference level representative of a population of subjects (e.g., a population of healthy subjects, a population of subjects clinically diagnosed with cellular or tissue damage or sterile inflammation, or a mixed population of healthy subjects and subjects clinically diagnosed with cellular or tissue damage or sterile inflammation). Various machine learning techniques may be used to produce a reference level using programs such as Scikit-learn Library in Python or Metaboanalyst®.

Any suitable laboratory techniques for determining the level of a biomarker (e.g., heme) in a biological sample can be used, including, but not limited, to flow cytometry (FC), fluorescence-activated cell sorting (FACS) Western blot, enzyme-linked immunosorbent assay (ELISA), mass spectrometry (MS), immunofluorescence (IF), immunoprecipitation (IP), radioimmunoassay, dot blotting, high performance liquid chromatography (HPLC), surface plasmon resonance, optical spectroscopy, and immunohistochemistry (IHC). For example, heme can be detected by chromatographic methods (e.g., normal-phase-high-performance liquid chromatography (NP-HPLC) or reversed-phase-HPLC (RP-HPLC)), capillary electrophoresis (CE) (e.g., chemiluminescence CE (CL-CE)), spectroscopic approaches (e.g., absorption spectroscopy, Raman spectroscopy), surface plasmon resonance (SPR) spectroscopy, fluorescence spectroscopy, or mass spectrometry (MS) (e.g., electrospray ionization (ESI)-MS (ESI-MS) or matrix-assisted laser desorption/ionization (MALDI)-MS)), enzyme-or protein-based methods for quantitative heme determination (e.g., heme quantification by peroxidase activity, fluorescence quenching, or by heme-binding single-domain antibodies), or using heme sensors.

In some embodiments, the biomarker is measured in a sample that was obtained from the patient no more than five hours (e.g., 1, 2, 3, 4, or 5 hours) after an administration of a therapeutic agent or a physical injury to the patient in the absence of bleeding. In some embodiments, the biomarker is measured in a sample that was obtained from the patient more than five hours (e.g., 5, 6, 7, 8, 9, 10, 11, 12, 13, 14, 15, 16, 17, 18, 19, 20, 21, 22, 23, or 24 hours) after an administration of a therapeutic agent or the occurrence of a physical injury to the patient in the absence of bleeding. In some embodiments, the biomarker is measured in a sample that was obtained from the patient one or more days (e.g., 1, 2, 3, 4, 5, 6, 7, 8, 9, 10, 11, 12, 13, or 14 days) after an administration of a therapeutic agent or a physical injury to the patient in the absence of bleeding.

A subject may be diagnosed as having cellular or tissue damage or sterile inflammation if the level of one or more biomarkers (e.g., heme) in a biological sample (e.g., a blood, serum, or plasma sample) obtained from the subject is substantially similar to a reference level of one or more of the same biomarkers (e.g., heme) derived from one or more subjects clinically diagnosed as having cellular or tissue damage or sterile inflammation (e.g., if the level of one or more biomarker (e.g., heme) in the subject is within 20%, e.g., +/−20%, 15%, 10%, 9%, 8%, 7%, 6%, 5%, 4%, 3%, 2%, 1%, or less, of a reference level of one or more of the same biomarkers (e.g., heme) derived from a positive control sample or population, e.g., one or more subjects clinically diagnosed as having cellular or tissue damage or sterile inflammation). A subject may also be diagnosed as having cellular or tissue damage or sterile inflammation if the level of one or more biomarkers (e.g., heme) obtained from the subject is substantially dissimilar to a reference level of one or more of the same biomarkers derived from one or more healthy subjects (e.g., if the level of one or more biomarkers in the subject deviates by more than 20%, e.g., +/−25%, 30%, 40%, 50%, 60%, 70%, 80%, 90%, 95%, 99%, or more, from a reference level of one or more of the same biomarkers derived from a negative control sample or population, e.g., one or more healthy subjects not having cellular or tissue damage or sterile inflammation).

A subject may be diagnosed as not having cellular or tissue damage or sterile inflammation (e.g., the subject may be determined to be healthy) if the level of one or more biomarkers (e.g., heme) in a biological sample (e.g., a blood, serum, or plasma sample, or other sample as described herein) obtained from the subject is substantially similar to a reference level of one or more of the same biomarkers derived from one or more healthy subjects (e.g., if the level of one or more biomarkers in the subject is within 20%, e.g., +/−20%, 15%, 10%, 9%, 8%, 7%, 6%, 5%, 4%, 3%, 2%, 1%, or less, of a reference level of one or more of the same biomarkers derived from a negative control sample or population, e.g., one or more healthy subjects not having cellular or tissue damage or sterile inflammation). A subject may also be diagnosed as not having cellular or tissue damage or sterile inflammation (e.g., as healthy) if the level of one or more biomarkers (e.g., heme) in a biological sample (e.g., a blood, serum, or plasma sample) obtained from the subject is substantially dissimilar to a reference level of one or more of the same biomarkers derived from one or more subjects clinically diagnosed as having cellular or tissue damage or sterile inflammation (e.g., if the level of one or more biomarkers in the subject deviates by more than 20%, e.g., +/−25%, 30%, 40%, 50%, 60%, 70%, 80%, 90%, 95%, 99%, or more, from a reference level of one or more of the same biomarkers derived from a positive control sample or population, e.g., one or more subjects clinically diagnosed as having cellular or tissue damage or sterile inflammation).

In some examples, heme is the selected biomarker and the heme levels are compared to a baseline heme level for the subject. Any suitable baseline heme level may be used. In some examples, a subject may have a baseline heme level of less than about 5 μM (e.g., about 1 μM or less) or about 5 μM to about 10 μM (e.g., about 5 μM, about 6 μM, about 7 μM, about 8 μM, about 9 μM, or about 10 μM). In some examples, the subject may be determined to have a heme level in a biological sample (e.g., blood, e.g., serum, or other biological sample described herein) of about 1,000 μM to about 50,000 μM (e.g., about 1,000 μM, about 2,000 μM, about 5,000 μM, about 6,000 μM, about 7,000 μM, about 8,000 μM, about 9,000 μM, about 10,000 μM, about 15,000 μM, about 20,000 μM, about 25,000 μM, about 30,000 μM, about 35,000 μM, about 40,000 μM, about 45,000 μM, or about 50,000 μM). A heme level of greater than about 5 μM to about 10 μM (e.g., greater than about 10 μM) measured in a biological sample from a subject indicates the presence of cellular or tissue damage, sterile inflammation, or the occurrence of a physical trauma in the subject. For example, detection (e.g., measurement) of a level of heme in a biological sample from a subject of about 1,000 μM to about 50,000 μM, relative to a baseline level of heme of about 5 μM to about 10 μM (e.g., from a healthy subject) indicates the presence of cellular or tissue damage, sterile inflammation, or the occurrence of a physical trauma in the subject.

The subject may be one who has experienced a blast injury (e.g., due to an explosion), as in the case of a first responder or military or law enforcement service member, or a rotational injury, such as an injury resulting from spinning, as in the case of a ballerina. The subject may also be one who has experienced a sports-related injury, such as an impact sustained during a contact sport, such as football, soccer, basketball, hockey, or rugby, or other sport, such as running, tennis, handball, and the like. An increase in heme levels (e.g., in a biological fluid, such as blood (e.g., plasma or serum), urine, CSF, etc.) may indicate the presence of a traumatic or physical injury in the subject, e.g., reflecting the presence of tissue damage in the subject (e.g., a concussion, contusion, sprain, strain, fracture, or joint dislocation).

Treatment of a Subject Determined to have Cellular or Tissue Damage

Provided herein are methods for treating a subject who is determined to have cellular or tissue damage based on elevated heme levels (e.g., a heme level of greater than about 10 μM, such as about 1,000 μM to about 50,000 μM) in a biological sample (e.g., a biological fluid, e.g., blood (e.g., plasma or serum), CSF, urine, wound fluid (e.g., wound drains), lung fluid (e.g., lung wash fluid collected from a bronchoalveolar lavage), abdomen fluid (e.g., ascites or fluid collected from peritoneal lavage), or sweat) obtained from the subject (e.g., relative to a reference level or a healthy subject). A subject may be selected for any treatment disclosed herein based on the presence of elevated heme levels in a biological sample obtained from the subject. Moreover, heme levels can be used to stratify or categorize the subject by risk level (e.g., a level of severity of cellular or tissue damage that has already occurred, that may occur, or is in the process of occurring). For example, heme levels in the blood of a subject may peak within a short period of time after an occurrence of cellular or tissue damage (e.g., within about 15 minutes, 30 minutes, 45 minutes, or 1 hour), such as after a surgical procedure, administration of a therapeutic agent (e.g., a chemotherapeutic agent, such as, e.g., cisplatin), or after a traumatic event, such as a physical injury, for example, a traumatic brain injury, e.g., a concussion. The heme levels in the blood of a subject may remain relatively high (e.g., about 1000 μM to about 4000 μM) for up to an hour or more (e.g., up to 2 weeks after an occurrence of cellular or tissue damage) and may transiently decrease thereafter. Thus, heme levels in the blood of a subject may be used to stratify or categorize a subject as one who has recently experienced, or is actively experiencing, cellular or tissue damage. Specifically, if heme levels in the blood of a subject are above a reference level (e.g., above a reference level of about 1 μM to about 10 μM, e.g., about 5 μM to about 10 μM, e.g., about 1 μM, about 2 μM, about 3 μM, about 4 μM, about 5 μM, about 6 μM, about 7 μM, about 8 μM, about 9 μM, or about 10 μM), then the subject has likely experienced, or is actively experiencing, cellular or tissue damage. Generally, a high level of heme in the blood, relative to a reference level, may indicate that an occurrence of cellular or tissue damage occurred recently (e.g., within minutes or hours after the sample from a subject was taken). If heme levels in the blood of a subject remain above a reference level for an extended period (e.g., 1 hour to 24 hours, e.g., 1 hour 6 hours, 12 hours, 18 hours, or 24 hours), then the subject may be determined to be actively experiencing cellular or tissue damage, A reduction in the level of heme in the blood of a subject (e.g., by over 50% or more over a period of time (e.g., over about 15 minutes to 3 hours, e.g., 15 minutes, 30 minutes, 45 minutes, 1 hour, 1.5 hours, or 2 hours) relative to an initial determination of the heme level may indicate that the subject is no longer experiencing an event causing cellular or tissue damage. A subject determined to be experiencing or to have experienced an event causing cellular or tissue damage can be treated by any of the methods described herein.

For example, a subject in need of treatment for, or prevention against cellular or tissue damage can be administered an effective amount of a therapeutic agent (e.g., a heme scavenger therapy, a CO therapy, or a combination thereof). For example, the subject may be determined in advance to have a level of heme in a biological sample from the subject that is at or above a reference level of heme (as described herein, such as above a level of about 10 μm).

In another example, the disclosure provides a method of treating or preventing sterile inflammation in a subject in need thereof by administering an effective amount of a therapeutic agent (e.g., heme scavenger therapy, a CO therapy, or a combination thereof) to the subject, in which the subject has been determined to have a level of heme in a biological sample (as described herein) that is at or above a reference level of heme (e.g., above a level of about 10 μm, such as a level in the range of about 1,000 μM to about 50,000 μM).

In some examples, the cellular or tissue damage occurs as a result of an ischemic event, hypofusion/reperfusion injury, treatment with a drug, such as chemotherapy, pancreatitis, a physical injury, such as a concussion, a drug overdose, surgery (e.g., open chest surgery (e.g., cardiopulmonary bypass surgery), hip replacement surgery, plastic surgery, etc.), hypotension, or shock. In some examples, the ischemic event comprises a stroke or a TIA. In some examples, the hypofusion/reperfusion injury comprises IRI. In some examples, the cellular or tissue damage comprises cardiotoxicity, liver toxicity, kidney toxicity, or brain toxicity. In some examples, the cellular or tissue damage comprises cardiotoxicity.

In some examples, the subject is undergoing treatment with a chemotherapeutic agent (e.g., the subject is a cancer patient). The subject may be undergoing treatment with any suitable chemotherapeutic agent. In some examples, the chemotherapeutic agent is an anthracycline or a platinum-based chemotherapeutic agent. In some examples, the anthracycline is doxorubicin. In some examples, the heme scavenger therapy, the CO therapy, or the combination thereof attenuates acute effects of doxorubicin treatment, chronic effects of doxorubicin treatment, or both, compared to a control therapy. In some examples, the acute effects of doxorubicin treatment comprise an increase in serum creatine kinase levels, heme levels, or both. In some examples, the chronic effects of doxorubicin treatment comprise long-term cardiac injury, an increase in a level of one or more cardiac dysfunction markers compared to a reference level of the one or more cardiac dysfunction markers, or both. In some examples, the cardiac dysfunction markers comprise ANP, BNP, β-MyHC, or a combination thereof. In some examples, the platinum-based chemotherapeutic agent is cisplatin, carboplatin, or oxaliplatin. In some examples, the platinum-based chemotherapeutic agent is cisplatin.

In some examples, the subject is undergoing treatment with a radiation therapy (e.g., internal beam radiation, external beam radiation therapy, brachytherapy, intraoperative radiation therapy (IORT), or stereotactic radiosurgery (SRS)) that causes cellular or tissue toxicity. In some examples, the subject may have experienced a radiation injury (e.g., an overexposure to x-rays, gamma rays, electron beams, or protons) resulting in cellular or tissue toxicity. Any suitable heme scavenger can be administered to the subject. In some examples, the heme scavenger therapy could include hemopexin, haptoglobin, albumin, α1-microglobulin, or α1-antitrypsin. In some examples, the heme scavenger therapy comprises hemopexin.

Any suitable CO therapy can be administered to the subject. In some examples, the CO therapy is a low dose CO therapy. In some examples, the CO therapy comprises HBI-002 or CO-306. The CO therapy may be administered by any suitable route, e.g., by enteral, gastrointestinal, or topical administration, or via inhalation by an aerosol spray, mist, or powder. For example, the CO therapy may be administered with a gas-entrapping material (e.g., a CO-hydrogel or foam), as is described in Byrne et al., Sci. Transl. Med., 14(651): eabl4135, 2022, which is incorporated herein by reference in its entirety.

Any of the therapeutic agents disclosed herein may be included in a pharmaceutical composition. Pharmaceutical compositions described herein may contain a therapeutic agent (e.g., a heme scavenger therapy, a CO therapy, or a combination thereof) described herein in combination with one or more pharmaceutically acceptable excipients. For instance, pharmaceutical compositions described herein can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions. The compositions can also be prepared so as to contain the active agent at a desired concentration. For example, a pharmaceutical composition described herein may contain at least 10% (e.g., 10%, 20%, 30%, 40%, 50%, 60%, 70%, 80%, 90%, 95%, 97%, 98%, 99%, 99.5%, 99.9%, or 100%) active agent by weight (w/w).

Additionally, an active agent that can be incorporated into a pharmaceutical formulation can itself have a desired level of purity. For example, a therapeutic agent (e.g., a heme scavenger therapy, a CO therapy, or a combination thereof) described herein may be characterized by a certain degree of purity. A therapeutic agent (e.g., a heme scavenger therapy, a CO therapy, or a combination thereof) described herein may be at least 10% pure prior to incorporating the antibody into a pharmaceutical composition (e.g., 10%, 20%, 30%, 40%, 50%, 60%, 70%, 80%, 90%, 95%, 97%, 98%, 99%, 99.5%, 99.9%, 99.99%, or 100% pure).

Pharmaceutical compositions of a therapeutic agent (e.g., a heme scavenger therapy) described herein can be prepared for storage as lyophilized formulations or aqueous solutions by mixing the antibody having the desired degree of purity with optional pharmaceutically acceptable carriers, excipients or stabilizers typically employed in the art, e.g., buffering agents, stabilizing agents, preservatives, isotonifiers, non-ionic detergents, antioxidants, and other miscellaneous additives. See, e.g., Remington's Pharmaceutical Sciences, 16th edition (Osol, ed. 1980; incorporated herein by reference). Such additives must be nontoxic to the recipients at the dosages and concentrations employed.

A therapeutic agent (e.g., a heme scavenger therapy, a CO therapy, or a combination thereof) described herein can be administered to a mammalian subject (e.g., a human) by a variety of routes, such as orally, transdermally, subcutaneously, intranasally, intravenously, intramuscularly, intraocularly, intratumorally, parenterally, topically, intrathecally and intracerebroventricularly, for the treatment of, e.g., the diseases and conditions described herein (e.g., cellular or tissue damage or sterile inflammation). The most suitable route for administration in any given case will depend on the particular agent administered, the patient, pharmaceutical formulation methods, administration methods (e.g., administration time and administration route), the patient's age, body weight, sex, severity of the diseases being treated, the patient's diet, and the patient's excretion rate.

A physician having ordinary skill in the art can readily determine an effective amount of a therapeutic agent (e.g., a heme scavenger therapy, a CO therapy, or a combination thereof) for administration to a mammalian subject (e.g., a human) in need thereof. For example, a physician could start prescribing doses of a therapeutic agent (e.g., a heme scavenger therapy, a CO therapy, or a combination thereof) described herein at levels lower than that required in order to achieve the desired therapeutic effect and gradually increase the dosage until the desired effect is achieved. Alternatively, a physician may begin a treatment regimen by administering a therapeutic agent (e.g., a heme scavenger therapy, a CO therapy, or a combination thereof) at a high dose and subsequently administering progressively lower doses until a therapeutic effect is achieved. In general, a suitable daily dose of a therapeutic agent (e.g., a heme scavenger therapy, a CO therapy, or a combination thereof) will be an amount of the compound which is the lowest dose effective to produce a therapeutic effect. A therapeutic agent (e.g., a heme scavenger therapy, a CO therapy, or a combination thereof) described herein may be administered, e.g., orally (e.g., by a drink), by injection, such as by intravenous, intramuscular, intraperitoneal, or subcutaneous injection, optionally proximal to the site of the target tissue. A daily dose of a therapeutic composition of a therapeutic agent (e.g., a heme scavenger therapy, a CO therapy, or a combination thereof) described herein may be administered as a single dose or as two, three, four, five, six or more doses administered separately at appropriate intervals throughout the day, week, month, or year, or as needed, optionally, in unit dosage forms. While it is possible for a therapeutic agent (e.g., a heme scavenger therapy, a CO therapy, or a combination thereof) described herein to be administered alone, it may also be administered as a pharmaceutical formulation in combination with excipients, carriers, and optionally, additional therapeutic agents.

The effective dose of a therapeutic agent (e.g., a heme scavenger therapy, a CO therapy, or a combination thereof) described herein can range, for instance, from about 0.0001 to about 100 mg/kg of body weight per single (e.g., bolus) administration, multiple administrations or continuous administration (e.g., a continuous infusion), or to achieve a serum concentration of 0.0001-5000 μg/mL serum concentration per single (e.g., bolus) administration, multiple administrations or continuous administration (e.g., continuous infusion), or any effective range or value therein depending on the condition being treated, the route of administration and the age, weight, and condition of the subject. In certain embodiments, each dose can range from about 0.0001 mg to about 500 mg/kg of body weight. For instance, a pharmaceutical composition described herein may be administered in a daily dose in the range of 0.001-100 mg/kg (body weight). The dose may be administered one or more times (e.g., 2-10 times) per day, week, month, or year to a mammalian subject (e.g., a human) in need thereof.

A therapeutic agent (e.g., a heme scavenger therapy, a CO therapy, or a combination thereof) can be administered to a patient by way of a continuous intravenous infusion or as a single bolus administration. The therapeutic agent (e.g., a heme scavenger therapy, a CO therapy, or a combination thereof) may be administered to a patient in an amount of, for example, from 0.01 μg to about 5 g in a volume of, for example, from 10 μL to 10 mL. The therapeutic agent (e.g., a heme scavenger therapy, a CO therapy, or a combination thereof) may be administered to a patient over the course of several minutes to several hours. For example, the therapeutic agent (e.g., a heme scavenger therapy, a CO therapy, or a combination thereof) may be administered to a patient over the course of from 5 minutes to 5 hours, such as over the course of 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 80 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, 185 minutes, 190 minutes, 195 minutes, 200 minutes, 205 minutes, 210 minutes, 215 minutes, 220 minutes, 225 minutes, 230 minutes, 235 minutes, 240 minutes, 245 minutes, 250 minutes, 255 minutes, 260 minutes, 265 minutes, 270 minutes, 275 minutes, 280 minutes, 285 minutes, 290 minutes, 295 minutes, or 300 minutes, or more.

A therapeutic agent (e.g., a heme scavenger therapy, a CO therapy, or a combination thereof) described herein may be administered in combination with one or more additional active agents. When an additional therapeutic agent is administered to a patient in combination with a therapeutic agent (e.g., a heme scavenger therapy, a CO therapy, or a combination thereof), the additional therapeutic agent may be administered to the patient by way of any suitable route, e.g., a single bolus administration or continuous intravenous infusion.

The therapeutic agent (e.g., a heme scavenger therapy, a CO therapy, or a combination thereof) described herein may be administered to the subject until the level of heme detected in a body fluid (e.g., blood or CSF) has decreased to 100 μM or less (e.g., 10 μM or less, such as, (e.g., 5 μM or less, for example 1 μM or less).

### EXAMPLES

### Example 1

### Heme as a Biomarker for Cytotoxicity During Chemotherapy

Doxorubicin (DOX) carries adverse effects such as cardiotoxicity^{1,2}. Iron plays a key role in DOX cardiotoxicity and free heme is toxic to cells and is a major source of redox-active iron^{3,4}. To limit free Heme toxicity, Heme is metabolized by Heme Oxygenase-1 (HO-1), a known cardioprotective enzyme, into iron, bilirubin, and carbon monoxide (CO). CO presents a promising approach to preventing anthracycline cardiotoxicity⁵⁻⁷. Inhaled CO or carrier molecule-bound CO, present substantial barriers to use. HBI-002 (Hillhurst Biopharmaceuticals, CA): oral CO drug produces COHb well below levels known to be associated with toxicity and amenable to administration of CO as a drink⁸.

This example evaluated the hypothesis that a sudden elevation in heme contributes to DOX-induced cardiotoxicity, and evaluated whether HBI-002 attenuates DOX-induced cardiotoxicity.

Surprisingly, we have discovered significant increases in heme levels after administration of the chemotherapeutic agents DOX and cisplatin. Based on these data, it is expected that any cytotoxic therapeutic agents will lead to release of heme, which can be used as a biomarker for cellular and tissue damage.

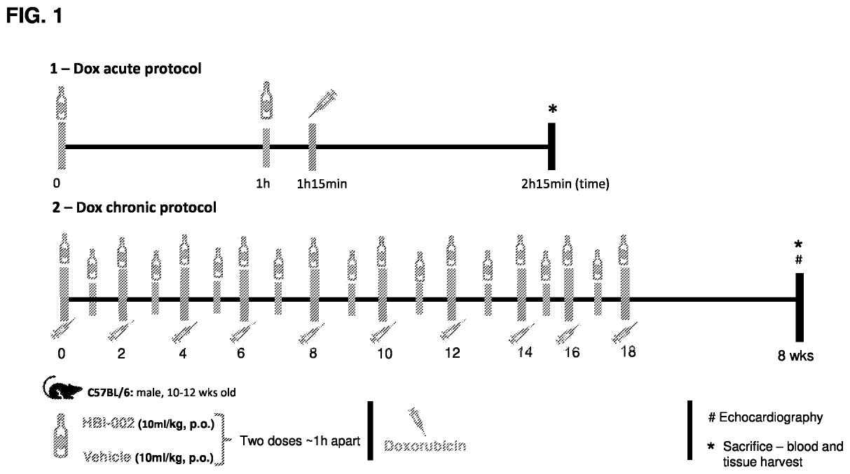

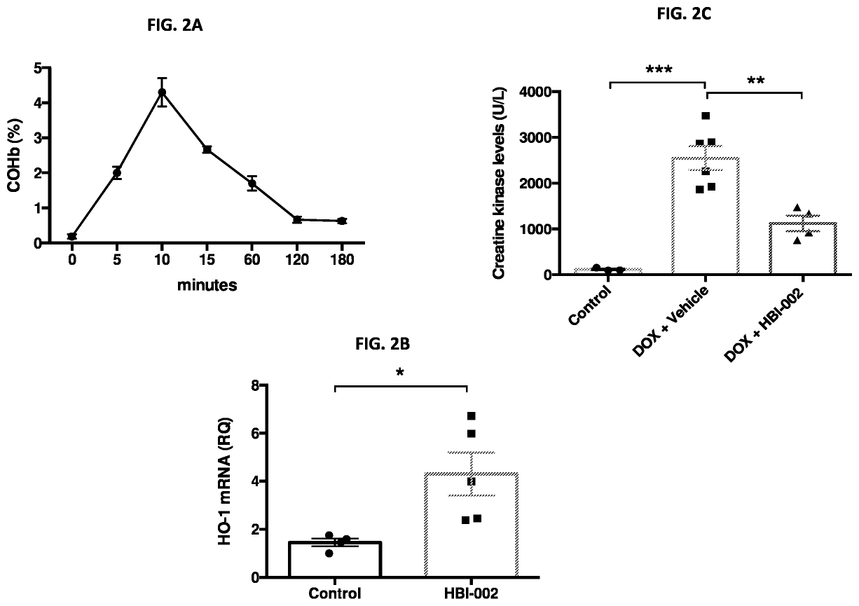

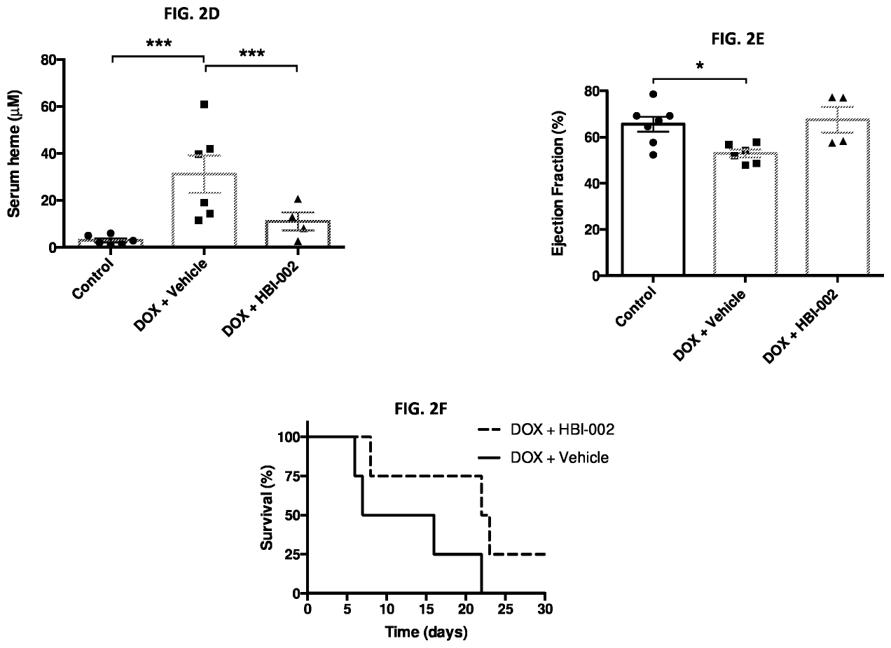

Male C57BL/6 mice (10-12 weeks old) were administered HBI-002 (10 ml/kg, per os) or a control vehicle (10 ml/kg, per os) at 0 hr and 1 hr, followed by administration of DOX at 1.25 hr (20 mg/kg, intraperitoneal injection) and blood and tissue collection at 2.25 hr (FIG. 1). HBI-002 prevented short and mid-term effects of single dose of DOX (FIGS. 2A-2F). Interestingly, we observed a significant increase in serum heme upon administration of DOX (FIG. 2D).

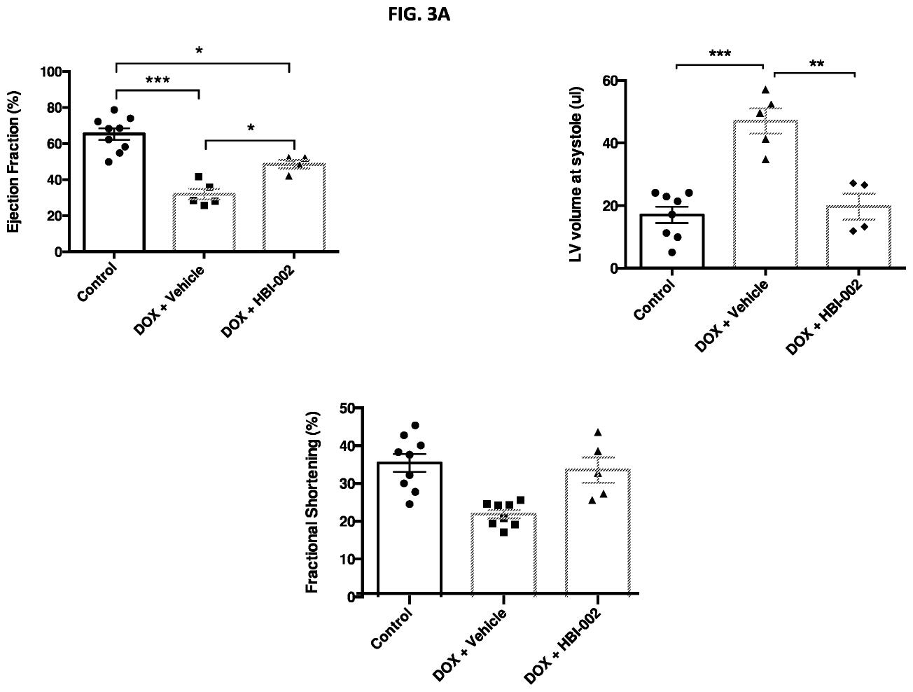

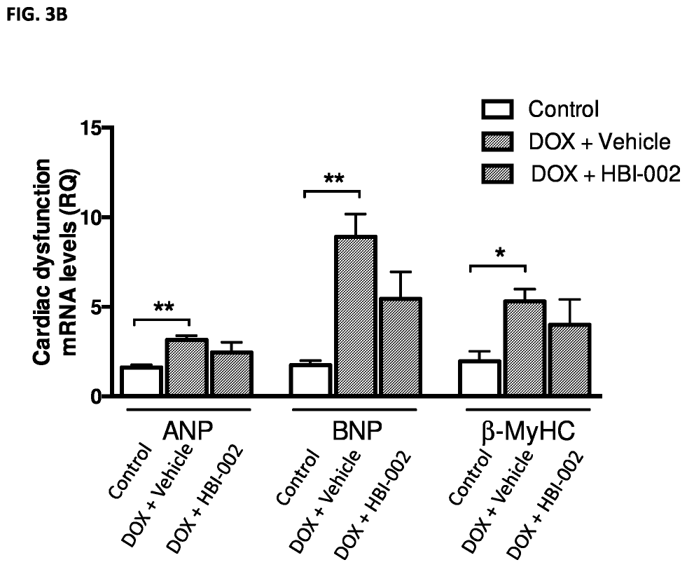

HBI-002 also attenuated long-term (8-weeks) cardiotoxic effects of DOX (FIGS. 3A and 3B). HBI-002 chronic dose regimen prevented long-term DOX-induced cardiac injury.

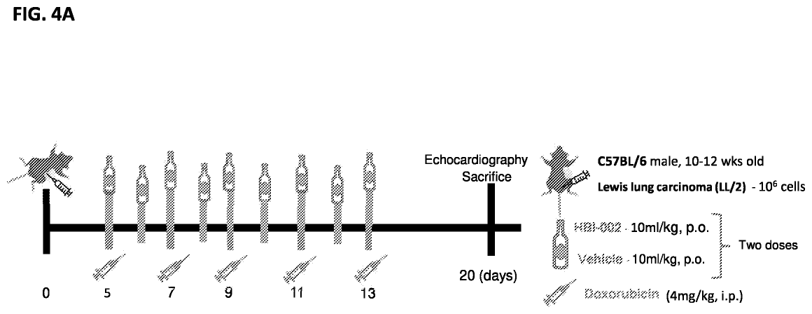

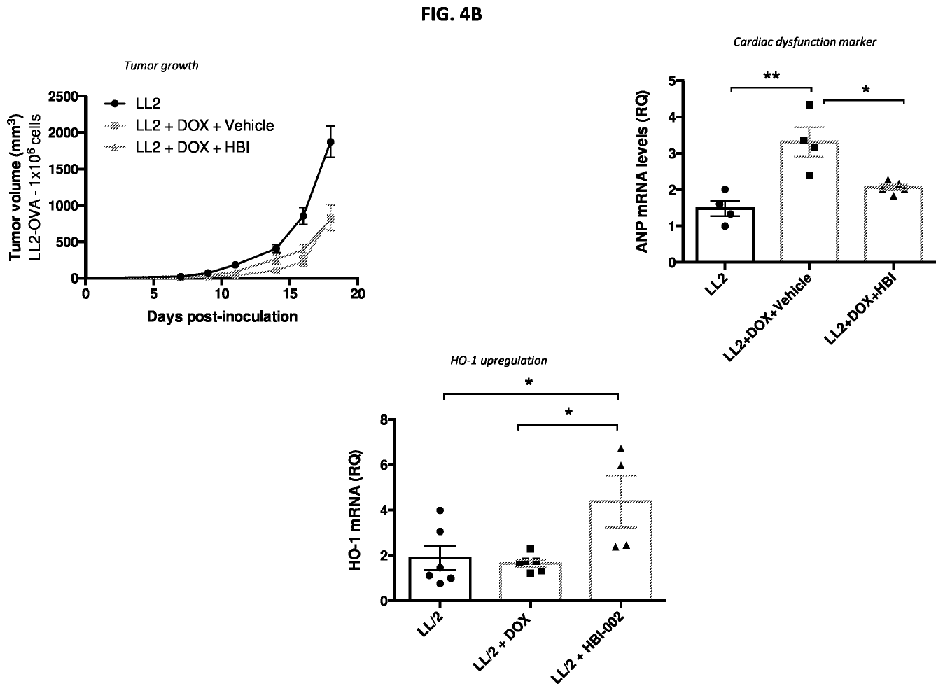

HBI-002 does not affect DOX chemotherapy and promotes cardioprotection, possibly by HO-1 overexpression in the heart (FIGS. 4A and 4B).

In conclusion, HBI-002 prevented acute muscle damage and serum heme release associated with DOX administration. HBI-002 also upregulated HO-1 expression in the heart. Further, HBI-002 can be administered in combination with DOX. The oral CO drink HBI-002, which is designed for use in hospitals and at home, may maintain the cancer therapeutic benefits of DOX and mitigate cardiac damage caused by DOX treatment.

Thus, we have established that the level of heme (e.g., in serum) can be used as a biomarker/diagnostic for cytotoxicity (e.g., cardiotoxicity), such as cytotoxicity resulting from chemotherapies (e.g., DOX or cisplatin).

### Example 2

### Use of Heme as a Biomarker for Sterile Inflammation or Sterile Injury

In this example, heme (e.g., extracellular heme, e.g., free heme) is used as a biomarker for sterile inflammation (e.g., sterile inflammation associated with an ischemic event (e.g., a stroke or TIA), hypofusion/reperfusion injury (e.g., IRI), chemotherapy, pancreatitis, a concussion, a drug overdose, surgery, hypotension, or shock). Typically, baseline levels of heme in blood or other biological fluids are low or undetectable. However, we have observed unexpectedly high levels of heme in the context of a number of different types of sterile inflammation, as discussed further below. In some examples, the level of heme reflects the inherent risk of further inflammation and/or tissue injury. This discovery was particularly unexpected because heme was not expected to be released as a result of sterile inflammation or sterile injury.

For example, we have found that heme levels are elevated in IRI, which is relevant to indications such as organ transplant, stroke, myocardial infarction, clotting emboli, and others. The elevation of heme levels was observed in a mouse model of kidney IRI. Therefore, elevated heme levels can be used as a biomarker to identify a patient having IRI.

In another example, we have found that heme levels are elevated following cardiac surgery, as assessed in a pig model. Therefore, elevated heme levels may be used as a biomarker for cellular or tissue damage due to surgery. For example, heme levels can be monitored over time to assess the success of the surgery; elevated heme levels may indicate the presence of tissue damage, while reduction in heme levels may indicate successful healing.

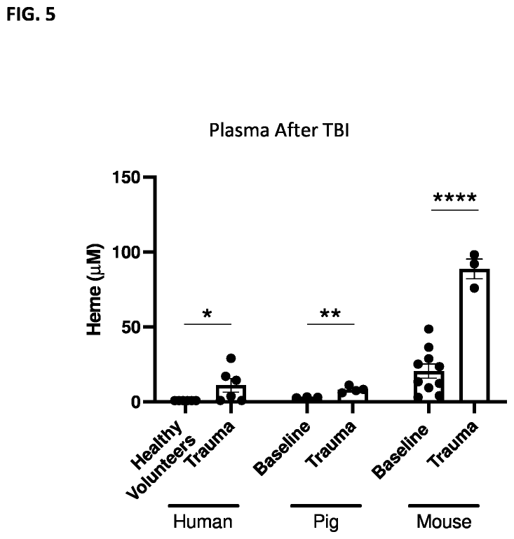

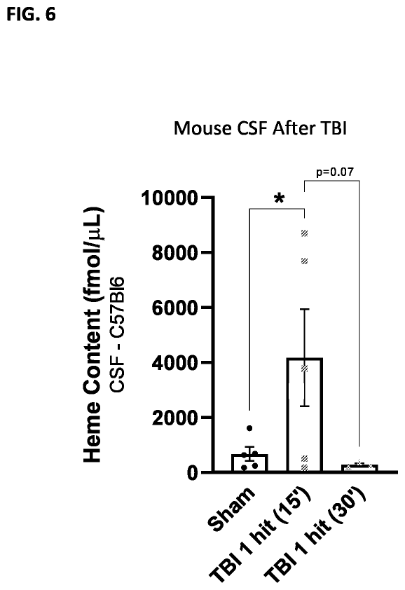

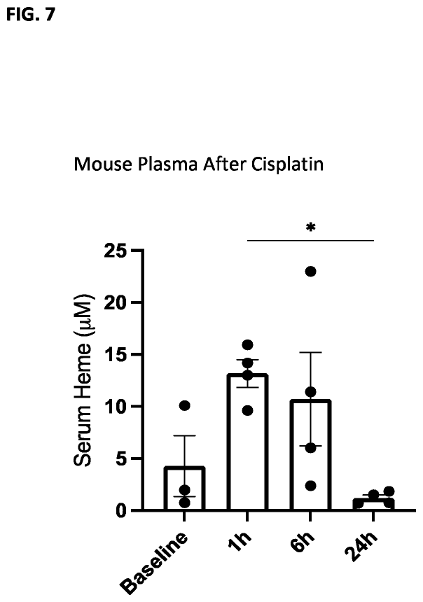

In another example, heme levels were used as a biomarker for a traumatic brain injury (TBI), e.g., a concussion. For example, we have observed unexpectedly elevated levels of heme in blood samples from mice, pigs, and humans who have experienced a TBI, e.g., a concussion (FIG. 5). Increased levels of heme were rapidly detected after the TBI, e.g., the concussion, e.g., within 1 hour (e.g., within 15 min and 30 min) following the concussion (FIG. 6). Based on these results, heme can be used as a biomarker for a TBI, e.g., a concussion or other traumatic injury. For example, a rapid heme test can be used to test a sample (e.g., a blood sample) from a patient suspected of having had a concussion or other traumatic injury, e.g., a player at a sporting event who has experienced an injury (e.g., a head injury). The patient may be tested within about 1 min, about 5 min, about 10 min, about 15 min, about 20 min, about 25 min, about 30 min, about 35 min, about 40 min, about 45 min, about 50 min, about 55 min, or about 60 min following the injury. Other biological samples may also be used, e.g., biological fluids such as CSF, sweat, and fluids obtained from oral or nasal swabs.

In another example, heme levels can be used as a biomarker for drug overdoses. For example, a sample from a patient suspected of having a drug overdose can be tested for heme levels. An increase in heme levels (e.g., in a biological fluid, such as blood (e.g., plasma or serum), urine, CSF, etc.) may indicate the presence of a drug overdose, e.g., reflecting the presence of tissue damage in the subject (e.g., liver damage).

In another example, heme levels can be used as a biomarker for tissue damage caused by a medication taken by a subject for a given condition (e.g., a chronic or acute condition). The subject may be taking the medication for any period, including for the lifetime of the subject. An increase in heme levels (e.g., in a biological fluid, such as blood (e.g., plasma or serum), urine, CSF, etc.) may indicate the presence of tissue damage in the subject.

In another example, heme levels can be used as a biomarker for a traumatic brain injury (TBI), such as those resulting from any impact to the head of a subject, as in the case of a first responder or military or law enforcement personnel exposed to a blast injury (e.g., caused by an explosion), such as an injury to the head. An increase in heme levels (e.g., in a biological fluid, such as blood (e.g., plasma or serum), urine, CSF, etc.) may indicate the presence of a TBI, e.g., reflecting the presence of tissue damage in the subject (e.g., a concussion or contusion).

In another example, heme levels can be used as a biomarker for rotational injuries, such as those resulting from spinning, as in the case of a ballerina. An increase in heme levels (e.g., in a biological fluid, such as blood (e.g., plasma or serum), urine, CSF, etc.) may indicate the presence of a rotational injury, e.g., reflecting the presence of tissue damage in the subject (e.g., a concussion-like injury to the brain).

In another example, heme levels can be used as a biomarker for sports-related injuries, such as impacts sustained during football, soccer, basketball, hockey, rugby, running, tennis, handball, and the like. An increase in heme levels (e.g., in a biological fluid, such as blood (e.g., plasma or serum), urine, CSF, etc.) may indicate the presence of a sports-related injury, e.g., reflecting the presence of tissue damage in the subject (e.g., a concussion, contusion, sprain, strain, fracture, or joint dislocation).

In some examples, the heme levels are compared to a baseline heme level for the patient. Any suitable baseline heme level may be used. In some examples, a patient may have a baseline heme level of about 5 μM to about 10 μM (e.g., about 5 μM, about 6 μM, about 7 μM, about 8 μM, about 9 μM, or about 10 μM). In some examples, the patient may have a heme level in a biological sample (e.g., blood, e.g., serum) of about 1,000 μM to about 50,000 μM (e.g., about 1,000 μM, about 2,000 μM, about 5,000 μM, about 6,000 μM, about 7,000 μM, about 8,000 μM, about 9,000 μM, about 10,000 μM, about 15,000 μM, about 20,000 μM, about 25,000 μM, about 30,000 μM, about 35,000 μM, about 40,000 μM, about 45,000 μM, or about 50,000 μM).

In some examples, the level of heme is correlated with injury severity. In other words, higher heme levels are associated with more severe injuries. Therefore, the level of heme in a sample from the patient may be used to assess injury severity. In one example, a rapid assay to detect heme in a biological sample (e.g., a biological fluid, such as peripheral blood (e.g., serum), urine, CSF, sweat, or other excretions (e.g., tissue-specific excretions)) can be used to assess the severity of tissue damage.

### Example 3

### Use of Heme as a Biomarker for Monitoring Treatment with Therapeutic Agents that Cause Cellular or Tissue Damage

Heme (e.g., extracellular heme, e.g., free heme) can be used as a biomarker to monitor treatment with a therapeutic agent that can cause or has a risk of causing cellular or tissue damage, e.g., chemotherapeutic agents or other cytotoxic agents. For example, a first biological sample (e.g., CSF, blood, urine, wound fluid, lung fluid, or abdomen fluid) may be obtained from a patient prior to administration of the therapeutic agent(s). Next, the therapeutic agent is administered to the patient, and a second biological sample may be obtained from the patient at a time point following administration of the therapeutic agent(s). If heme levels in the second biological sample are elevated compared to heme levels in the first biological sample, the clinician may tailor the dosage of the therapeutic agent(s) based on the patient's heme levels (e.g., the dose level may be reduced over one or more doses). The heme level can be monitored following the dose adjustment, in which a reduced level of heme in the biological sample indicates a reduction in the level or severity of cellular or tissue damage. Alternatively, the clinician may discontinue the therapy or select additional or alternative therapeutic agents based on the patient's heme levels. Free heme is potentially cytotoxic as a Danger Associated Molecular Pattern molecule that can enhance inflammation and act to increase oxidative stress. Another option would be to continue administration of the therapy (at the same or a modified (e.g., reduced) dosage and in combination with a second therapy that includes a heme scavenger (e.g., hemopexin) or a CO therapy (e.g., HBI-002 or CO-306). Administration of the CO therapy to the patient can be monitored (e.g., in a biological sample (e.g., a blood sample, e.g., serum) from the subject) to ensure a reduction of elevated heme levels relative to a baseline level (e.g., a baseline level indicative of the absence of cellular or tissue injury).

### Example 4

### Measurement of Heme Levels

Any suitable approach can be used for measurement of heme (e.g., extracellular heme, e.g., free heme) levels in biological samples (e.g., a biological fluid, e.g., cerebrospinal fluid (CSF), urine, wound fluid (e.g., wound drains), lung fluid (e.g., lung wash fluid collected from a bronchoalveolar lavage), abdomen fluid (e.g., ascites or fluid collected from peritoneal lavage), or sweat) obtained from patients, e.g., for any of the methods and examples disclosed herein. For example, heme can be detected by chromatographic methods (e.g., normal-phase-high-performance liquid chromatography (NP-HPLC) or reversed-phase-HPLC (RP-HPLC)), capillary electrophoresis (CE) (e.g., chemiluminescence CE (CL-CE)), spectroscopic approaches (e.g., absorption spectroscopy, Raman spectroscopy), surface plasmon resonance (SPR) spectroscopy, fluorescence spectroscopy, or mass spectrometry (MS) (e.g., electrospray ionization (ESI)-MS (ESI-MS) or matrix-assisted laser desorption/ionization (MALDI)-MS)), enzyme-or protein-based methods for quantitative heme determination (e.g., heme quantification by peroxidase activity, fluorescence quenching, or by heme-binding single-domain antibodies), or using heme sensors.

### Example 5

### Use of Heme as a Biomarker for Monitoring Treatment with Cisplatin

Heme (e.g., extracellular heme, e.g., free heme) can be used as a biomarker to monitor treatment with cisplatin. For example, we administered 20 mg/kg of cisplatin by intraperitoneal (i.p.) injection to mice. Blood was collected either 1 hour, 6 hours, or 24 hours after cisplatin administration. Blood heme level was observed to increase at the 1 hour and 6 hour timepoint, but was significantly reduced after 24 hours, indicating that heme concentrations can be used as a biomarker for treatment with cisplatin (FIG. 7).

Example 6. Measurement of Heme Levels in Pigs After Surgery

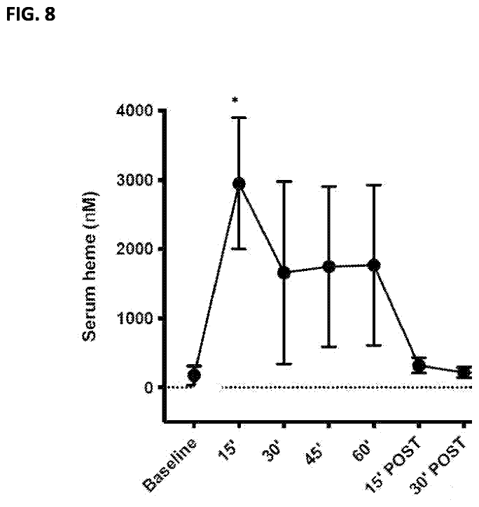

Heme (e.g., extracellular heme, e.g., free heme) was used as a biomarker to monitor inflammation caused by a scheduled surgical trauma in pigs. In brief, plasma from the pig was collected over the course of 1 hour during sternotomy and isolation of the left descending artery prior to ligation. A rapid increase in serum heme was observed during the first 15 minutes of surgery, followed by a decline in heme over time (FIG. 8). These results confirm that heme can be used as a biomarker for inflammation caused by trauma, e.g., trauma resulting from surgery (e.g., open chest surgery, hip replacement surgery, plastic surgery, etc.). These results further indicate that heme may also be measured in biological fluids obtained from a subject after a surgery, e.g., in plasma, whole blood, CSF, urine, wound fluid (e.g., surgical drains from wounds), lung fluid (e.g., lung wash fluid collected from a bronchoalveolar lavage), abdomen fluid (e.g., ascites or fluid collected from peritoneal lavage), or sweat.

### REFERENCES

1. Carvalho, F. S. et al. Doxorubicin-Induced Cardiotoxicity: From Bioenergetic Failure and Cell Death to Cardiomyopathy. Medicinal Research Reviews 34, 106-135 (2014).
2. Chatterjee, K., Zhang, J., Honbo, N. & Karliner, J. S. Doxorubicin cardiomyopathy. Cardiology 115, 155-162 (2010).
3. Gammella E, Maccarinelli F, Buratti P, Recalcati S, Cairo G. The role of iron in anthracycline cardiotoxicity. Frontiers in Pharmacology. 2014;5 FEB(February): 1-6.
4. Khechaduri A, Bayeva M, Chang HC, Ardehali H. Heme levels are increased in human failing hearts. Journal of the American College of Cardiology. 2013;61 (18): 1884-1893.
5. Zhao, S. et al. Carbon monoxide releasing molecule-2 attenuated ischemia/reperfusion-induced apoptosis in cardiomyocytes via a mitochondrial pathway. Molecular Medicine Reports 9, 754-762 (2014).
6. Soni, H. et al. Beneficial effects of carbon monoxide-releasing molecule-2 (CORM-2) on acute doxorubicin cardiotoxicity in mice: Role of oxidative stress and apoptosis. Toxicology and Applied Pharmacology 253, 70-80 (2011).
7. Suliman, H. B. et al. The CO/HO system reverses inhibition of mitochondrial biogenesis and prevents murine doxorubicin cardiomyopathy. Journal of Clinical Investigation 117, 3730-3741 (2007).
8. Belcher J.D., et al. Oral carbon monoxide therapy in murine sickle cell disease: Beneficial effects on vaso-occlusion, inflammation and anemia. PLOS One. 2018 Oct. 11;13 (10): e0205194.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations following, in general, the principles and including such departures from the invention that come within known or customary practice within the art to which the invention pertains and may be applied to the essential features hereinbefore set forth, and follows in the scope of the claims.

Other embodiments are within the claims.

1. A method of assessing cellular or tissue damage in a patient, the method comprising:
(a) determining a level of heme in a biological sample obtained from a patient; and
(b) identifying the patient as having cellular or tissue damage based on the level of heme in the biological sample from the patent, wherein a level of heme in the biological sample that is at or above a reference level of heme indicates that the patient has cellular or tissue damage.

2. A method of identifying a patient having sterile inflammation, the method comprising:
(a) determining a level of heme in a biological sample obtained from a patient; and
(b) identifying the patient as having sterile inflammation based on the level of heme in the biological sample from the patent, wherein a level of heme in the biological sample that is at or above a reference level of heme indicates that the patient has sterile inflammation.

3. A method of assessing sterile inflammation in a patient, the method comprising:
(a) obtaining a biological sample from the patient no more than five hours after administration of a therapeutic agent or a physical injury to the patient in the absence of bleeding; and
(b) detecting the level of heme in the biological sample.

4. The method of any one of claims 1-3, wherein the patient has experienced cellular or tissue damage.

5. The method of claim 4, wherein the cellular or tissue damage occurs as a result of an ischemic event, hypofusion/reperfusion injury, chemotherapy, radiation therapy, radiation injury, pancreatitis, a concussion, a drug overdose, surgery, hypotension, or shock.

6. The method of claim 5, wherein the ischemic event comprises a stroke or a transient ischemic attack (TIA).

7. The method of claim 5, wherein the hypofusion/reperfusion injury comprises ischemia reperfusion injury (IRI).

8. The method of claim 5, wherein the drug overdose is caused by a therapeutic agent that causes cellular or tissue toxicity.

9. The method of claim 8, wherein the drug is acetaminophen.

10. The method of any one of claims 1-9, wherein the level of heme in the biological sample from the patient indicates the severity of cellular or tissue damage in the patient.

11. The method of claim 1-10, wherein the patient is undergoing a treatment with a therapeutic agent that causes cellular or tissue toxicity.

12. A method of monitoring a patient undergoing a treatment with a therapeutic agent that causes cellular or tissue toxicity, the method comprising:
(a) determining a level of heme in a biological sample obtained from the patient at a time point during or after administration of the therapeutic agent; and
(b) comparing the level of heme in the biological sample from the patient with a reference level of heme, thereby monitoring the patient undergoing treatment with the therapeutic agent.

13. The method of claim 11 or 12, wherein the patient has an increase in the level of heme compared to the reference level of heme, and the treatment is adjusted or stopped.

14. The method of any one of claims 11-13, wherein the therapeutic agent comprises a chemotherapeutic agent.

15. The method of claim 14, wherein the chemotherapeutic agent comprises an anthracycline or a platinum-based chemotherapeutic agent.

16. The method of claim 15, wherein the anthracycline is doxorubicin.

17. The method of claim 15, wherein the platinum-based chemotherapeutic agent is cisplatin, carboplatin, or oxaliplatin.

18. The method of claim 17, wherein the platinum-based chemotherapeutic agent is cisplatin.

19. The method of any one of claims 1-18, wherein the method further comprises selecting a therapy for the patient to ameliorate the cellular or tissue damage.

20. The method of claim 19, wherein the therapy comprises a heme scavenger therapy, a carbon monoxide (CO) therapy, or a combination thereof.

21. The method of any one of claims 1-20, wherein the method further comprises administering an effective amount of a heme scavenger therapy, a CO therapy, or a combination thereof to the patient.

22. A method of treating or preventing cellular or tissue damage in a patient in need thereof, the method comprising administering an effective amount of a heme scavenger therapy, a CO therapy, or a combination thereof to the patient, wherein the patient has been determined to have a level of heme in the biological sample that is at or above a reference level of heme.

23. A method of treating or preventing sterile inflammation in a patient in need thereof, the method comprising administering an effective amount of a heme scavenger therapy, a CO therapy, or a combination thereof to the patient, wherein the patient has been determined to have a level of heme in the biological sample that is at or above a reference level of heme.

24. The method of claim 23, wherein the patient has experienced cellular or tissue damage.

25. The method of claim 22 or 24, wherein the cellular or tissue damage occurs as a result of an ischemic event, hypofusion/reperfusion injury, chemotherapy, radiation therapy, radiation injury, pancreatitis, a concussion, a drug overdose, surgery, hypotension, or shock.

26. The method of claim 25, wherein the ischemic event comprises a stroke or a TIA.

27. The method of claim 25, wherein the hypofusion/reperfusion injury comprises IRI.

28. The method of any one of claim 22 or 25-27, wherein the cellular or tissue damage comprises cardiotoxicity, liver toxicity, kidney toxicity, or brain toxicity.

29. The method of claim 28, wherein the cellular or tissue damage comprises cardiotoxicity.

30. The method of any one of claims 19-29, wherein the patient is undergoing treatment with a chemotherapeutic agent.

31. The method of claim 30, wherein the chemotherapeutic agent is an anthracycline or a platinum-based chemotherapeutic agent.

32. The method of claim 31, wherein the anthracycline is doxorubicin.

33. The method of claim 32, wherein the heme scavenger therapy, the CO therapy, or the combination thereof attenuates acute effects of doxorubicin treatment, chronic effects of doxorubicin treatment, or both, compared to a control therapy.

34. The method of claim 33, wherein the acute effects of doxorubicin treatment comprise an increase in serum creatine kinase levels, heme levels, or both.

35. The method of claim 33, wherein the chronic effects of doxorubicin treatment comprise long-term cardiac injury, an increase in a level of one or more cardiac dysfunction markers compared to a reference level of the one or more cardiac dysfunction markers, or both.

36. The method of claim 35, wherein the cardiac dysfunction markers comprise atrial natriuretic peptide (ANP), brain natriuretic peptide (BNP), cardiac beta-myosin heavy chain (β-MyHC), or a combination thereof.

37. The method of claim 31, wherein the platinum-based chemotherapeutic agent is cisplatin, carboplatin, or oxaliplatin.

38. The method of claim 37, wherein the platinum-based chemotherapeutic agent is cisplatin.

39. The method of any one of claims 20-38, wherein the CO therapy results in an elevated expression level of heme oxygenase-1 (HO-1) in the heart compared to a control therapy.

40. The method of any one of claims 19-39, wherein the heme scavenger therapy comprises hemopexin, haptoglobin, albumin, α1-microglobulin, or α1-antitrypsin.

41. The method of claim 40, wherein the heme scavenger therapy comprises hemopexin.

42. The method of any one of claims 19-41, wherein the CO therapy is a low dose CO therapy.

43. The method of any one of claims 19-42, wherein the CO therapy comprises HBI-002 or CO-306.

44. The method of any one of claims 19-43, wherein the heme scavenger therapy, the CO therapy, or the combination thereof is administered orally, by inhalation, intravenously, subcutaneously, topically, or intramuscularly.

45. The method of claim 44, wherein the heme scavenger therapy, the CO therapy, or the combination thereof is administered orally.

46. The method of any one of claims 1-45, wherein the biological sample is a biological fluid.

47. The method of claim 46, wherein the biological fluid is a blood sample, a cerebrospinal fluid (CSF) sample, urine, sweat, wound fluid, lung fluid, or abdomen fluid.

48. The method of claim 47, wherein the blood sample is a whole blood sample, a serum sample, a plasma sample, or a combination thereof.

49. The method of claim 48, wherein the blood sample is a serum sample.

50. The method of any one of claims 1-49, wherein the level of heme is the level of free heme.

51. The method of any one of claims 1-50, wherein the reference level of heme is a baseline level of heme.

52. The method of claim 51, wherein the baseline level of heme is a level of heme in a biological sample obtained from the patient prior to the onset of cellular or tissue damage.

53. The method of claim 51 or 52, wherein the reference level of heme is about 5 μM or above.

54. The method of claim 53, wherein the reference level of heme is about 5 μM to about 10 μM.

55. The method of any one of claims 1-54, wherein the patient has a level of heme of about 1000 μM or above.

56. The method of claim 55, wherein the patient has a level of heme of about 1000 μM to about 50,000 μM.

57. The method of any one of claims 1-56, wherein the biological sample is obtained from the patient within about 5 hours from the onset of cellular or tissue damage.

58. The method of claim 57, wherein the biological sample is obtained from the patient within about 1 minute to about 1 hour from the onset of cellular or tissue damage.

59. The method of claim 58, wherein the biological sample is obtained from the patient within about 30 min from the onset of cellular or tissue damage.

60. A kit for assessing cellular or tissue damage in a patient, the kit comprising:
(a) reagents for determining a level of heme in a biological sample obtained from a patient; and
(b) instructions to identify the patient as having cellular or tissue damage based on the level of heme in the biological sample from the patent, wherein a level of heme in the biological sample that is at or above a reference level of heme indicates that the patient has cellular or tissue damage.

61. A kit for identifying a patient having sterile inflammation, the kit comprising:
(a) reagents for determining a level of heme in a biological sample obtained from a patient; and
(b) instructions to identify the patient as having sterile inflammation based on the level of heme in the biological sample from the patent, wherein a level of heme in the biological sample that is at or above a reference level of heme indicates that the patient has sterile inflammation.
